(19) United States
(12) Patent Application Publication
(10) Pub. No.: US 2024/0254428 A1
(43) Pub. Date: Aug. 1, 2024

(54) SYSTEM AND METHOD FOR HIGH-THROUGHPUT PRECISION ROBOTIC EMBRYO MANIPULATION UNDER COMPUTER CONTROL AT LARGE-SCALE WITH HIGH RELIABILITY MICRODOSING

(71) Applicant: Cody Gilleland, Ph.D., Garland, TX (US)

(72) Inventor: Cody Gilleland, Ph.D., Garland, TX (US)

(21) Appl. No.: 18/564,036

(22) PCT Filed: May 27, 2022
(86) PCT No.: PCT/US2022/031382

§ 371 Date: Nov. 24, 2023

Related U.S. Application Data
(60) Provisional application No. 63/193,870, filed on May 27, 2021.

(51) Int. Cl. C12M 1/36; C12M 1/00; C12M 1/34; C12N 15/89; G16B 40/10

(52) U.S. Cl. CPC C12M 41/48; C12M 41/06; C12M 41/14; C12M 41/36; C12N 15/89; G16B 40/10

(57) ABSTRACT

In the present invention the robotic microinjection platform construction is provided in which three cameras enable simultaneous video streaming of high magnification, low magnification camera views with a third horizon view for Z-depth information to enable microneedle alignment. The triple camera method in combination with image processing algorithms enables automated microneedle alignment and organism targeting in low magnification followed by microneedle manipulations of the organism in high magnification. The object detection algorithms are trained using neural networks on large image datasets. The object detection algorithms improve over time as the training dataset grows as data is acquired with more experimentation.

### RELATED APPLICATIONS

This application claims the benefit of priority under 35 U.S.C. § 119(e) to U.S. Provisional Application No. 63/193,870, filed May 27, 2021 which is incorporated herein by reference in its entirety.

### FIELD AND BACKGROUND OF THE INVENTION

The present invention relates to biological laboratory instrumentation for gene editing, and more particularly the production of genetically engineered multicellular organisms at industrial scales with extreme reliability and consistency. We describe here a fully robotic platform and multi-view camera software interface for scalable transgenesis and cellular biopsy for genetic screening under computer software control that does not rely on hand dexterity since these methods do not require handling of organisms by a human operator. The present invention also enables precision biopsy of single or multiple biological cells and intracellular components after delicately penetrating through the outer tissue, cuticle, chorion, or zona pellucida layers to access the target cells of interest. The delicacy and high value of rare samples necessitates the presented invention to perform micromanipulations with extreme reliability and at industrial scales.

Microinjection is a commonly used transgenic technology to deliver genetic constructs to produce genetically engineered organisms where thick cuticles, chorions or outer casings prevent the use of gene gun or electroporation. This immensely useful technique has led to many scientific breakthroughs but it is limited in scale and precision by human dexterity, fatigue and the need for extensive training cycles. In the case of mosquitoes, manually handling the embryos to align or mount them for microinjection presents opportunity for accidental damage to the organism and is limited by skilled dexterity over long durations promoting human fatigue. Within the USA roughly only 10 people are sufficiently skilled to reliably produce transgenic Anopheles gambiae, the main malaria transmitting mosquito. The training process can take 9 months or more of practice before producing a single transgenic line. Manual methods for transgenesis require extensive training and are limited in scale by human fatigue since a single transgenic line can require over 15,000 microinjections into Anopheles gambiae embryos.

Prior to microinjection a human must position and orient the organism manually using a small pick or brush presenting an opportunity to physically damage the organism. This is a major rate-limiting bottleneck in speed and survivability for the organisms. Prior to manually handling the user must wait for the organism to develop (melanization and chorion hardening) and become rigid enough to avoid excessive damage. As time passes the organism progresses developmentally contributing to mosaicism as the genetic construct is injected after many nuclear and cellular divisions have already occurred. The more rigid outer layer (chorion) becomes more difficult to penetrate as time passes contributing to quartz microneedle breakage limiting scale and delicacy of microinjections due to a blunt microneedle tip. The blunt needle opening also requires the user to manually adjust the pressure level and duration each time the needle opening changes. Many organisms are lost due to excessive pressure pulses before the pressure can be adjusted to account for needle tip breakage.

Manual microneedle alignment is a rate-limiting step for installing new microneedles with different genetic constructs or replacing microneedles with clogged or broken tips. The current state of the art microscope requires that the user manually align the microneedle into the view of the high magnification objective. The user first switches to low magnifications for coarse XY positioning of the needle then manually changes the objective to the high magnification objective. The needle is then manually lowered blindly toward the high magnification objective. This can result in needle breakage which also damages the expensive microscope lens. For reference, a 20× high magnification objective presents a small target for visualizing the needle and can be easily missed by the operator. The 20× high magnification objectives typically have the following characteristics: working distance of ˜1 mm, a Z-focal depth of ˜2 μm, XY-field of view ˜200 μm×˜200 μm. The user must move slowly and carefully to prevent microneedle breakage by overshooting with a high failure rate. The rotation of a turret to change objective magnifications does now allow the objective to return to the original position with errors of several microns in each of the XYZ directions. Mechanizing the objective turret using a motor also creates a more complicated system that also drives up the cost of such a system while increasing the instrumentation footprint.

Using a piezoelectric actuator to vibrate the microneedle has shown to enhance penetration of the organism but has not been combined with high-pressure pulses without needle drift and parasitic off-axis vibration of several microns. The current state of the art designs require glue to attach the piezoelectric actuator to a metal holder. A pressurized tube protruding from the actuated end of the piezoelectric unit and the method for attaching the pressure source contributes to off-axis pulsation and XYZ positional drift which can lead to excessive damage during microinjection.

The limitations of manual microinjection are as follows: The fluid containing the genetic construct flows into the organism through the needle tip via a pressurized source with an on/off solenoid valve. The user must manually activate the injection pressure using a foot pedal or other signaling button. This is typically open-loop control of a manually preset pressure value (PSI) for a preset time duration (seconds). There is no rapid video feedback with an image processing algorithm to quickly adjust the level of pressure or a custom designed pressure wave to best fill the organism without causing collateral damage. Solenoid valves drive high currents and degrade over time due to electromagnetic coil burnout causing reliability issues of the valve itself and the high electrical current relay driver circuit. The abrupt opening and closing of solenoid valves cause internal damage to the organisms from the high-pressure changes due to cavitation from the water hammer effect to delicate developing organisms. If a needle becomes clogged the user must remove the clogged needle, fill a new needle with genetic construct, install the needle, and manually realign the needle causing significant delay of several minutes. Microneedles are pulled with a thin, closed tip that must be broken or beveled to open tip resulting in a large tip (˜10 microns) with high variability from each breaking or beveling step. The needle tip can also break during microinjection operation causing overfilling and damage to the delicate organism. The user must manually adjust the pulse pressure to react to the changes in tip size by turning a manual knob. The manual interactions limit scale, precision and delicacy while increasing user fatigue.

### SUMMARY OF THE INVENTION, LIST OF DRAWINGS AND DETAILED DESCRIPTION

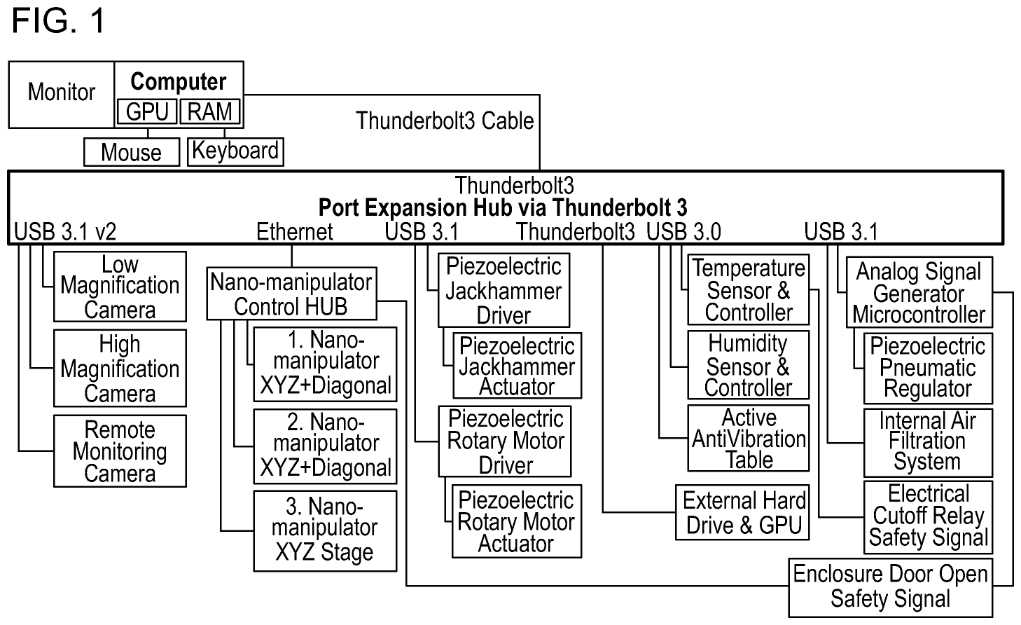

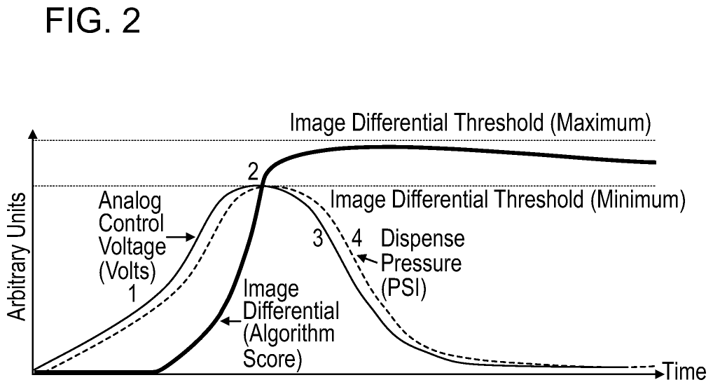

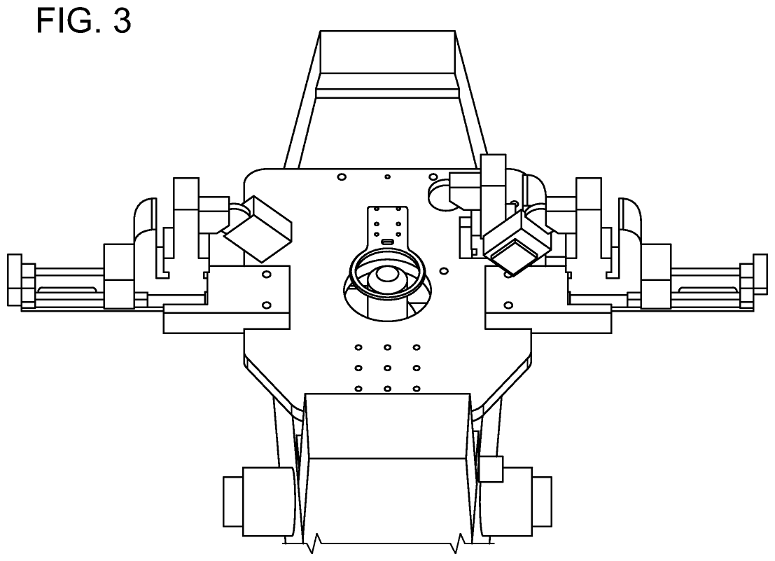

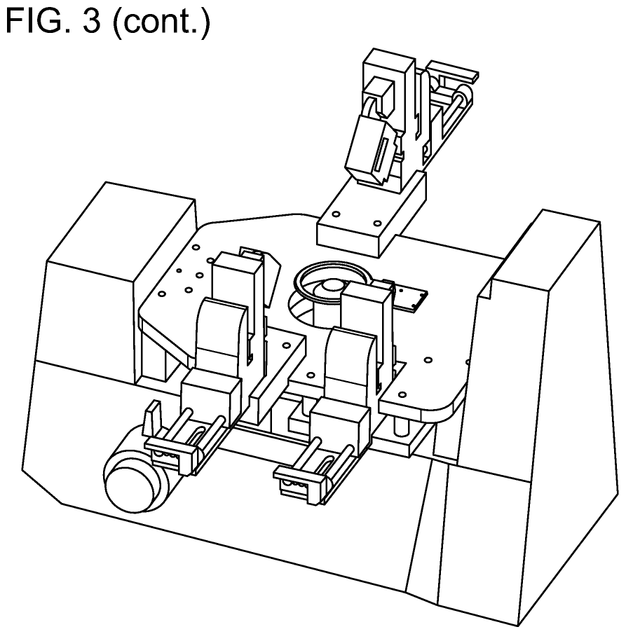

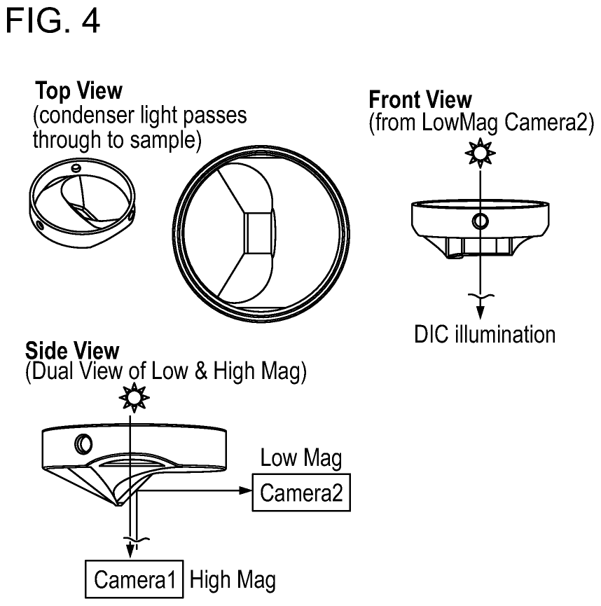

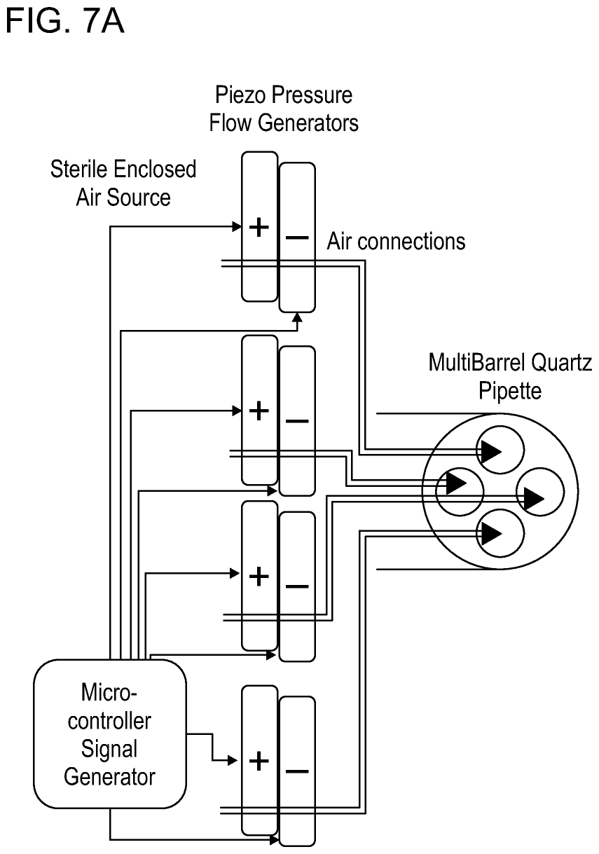

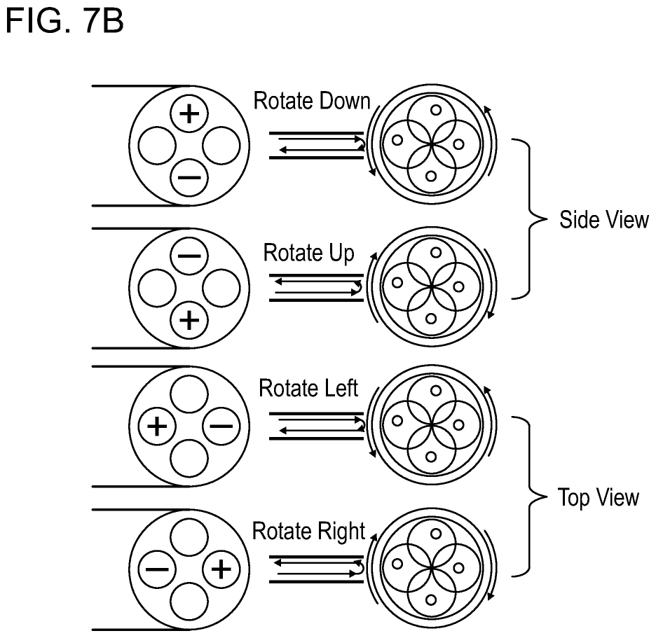

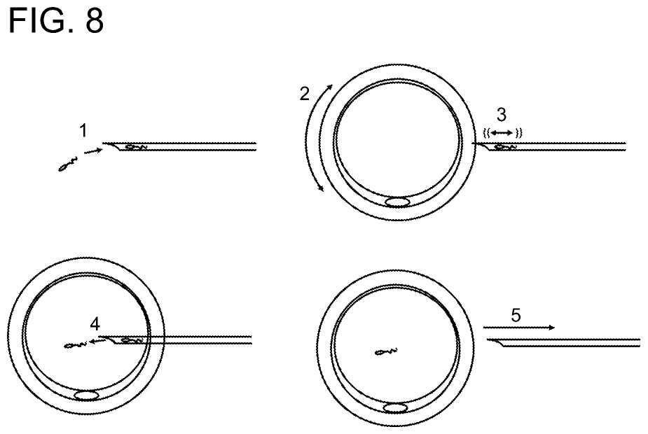

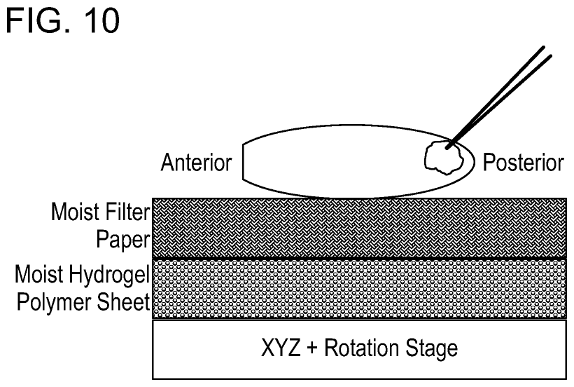

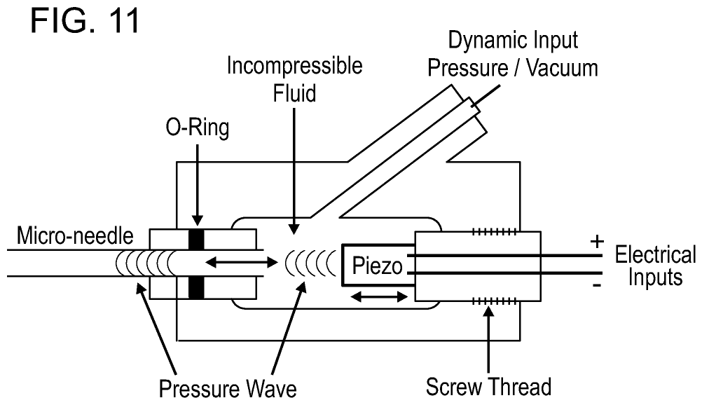

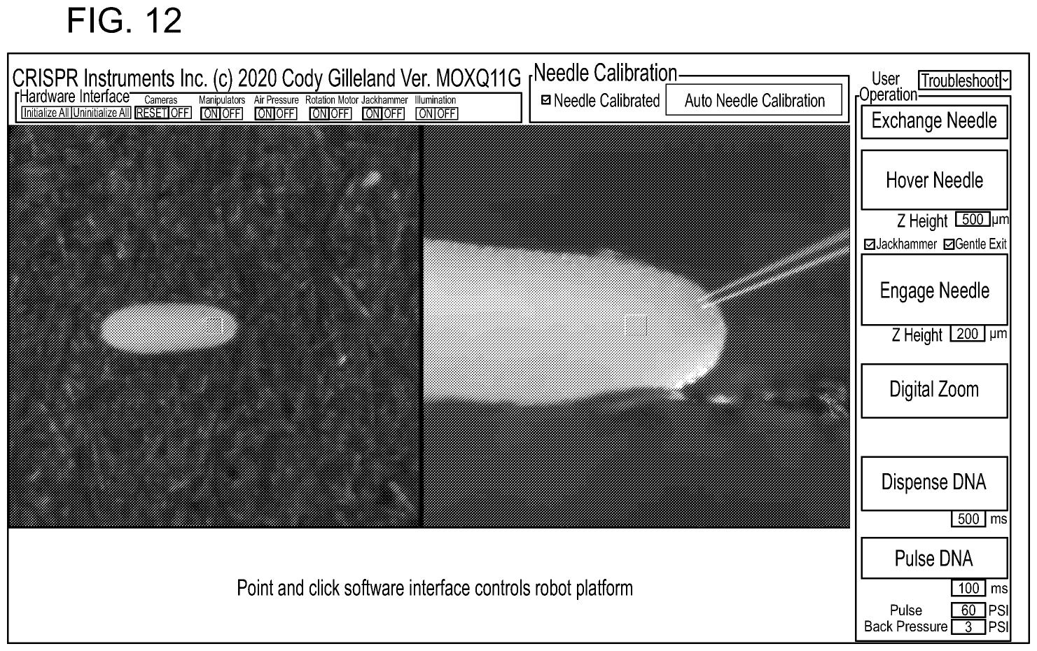

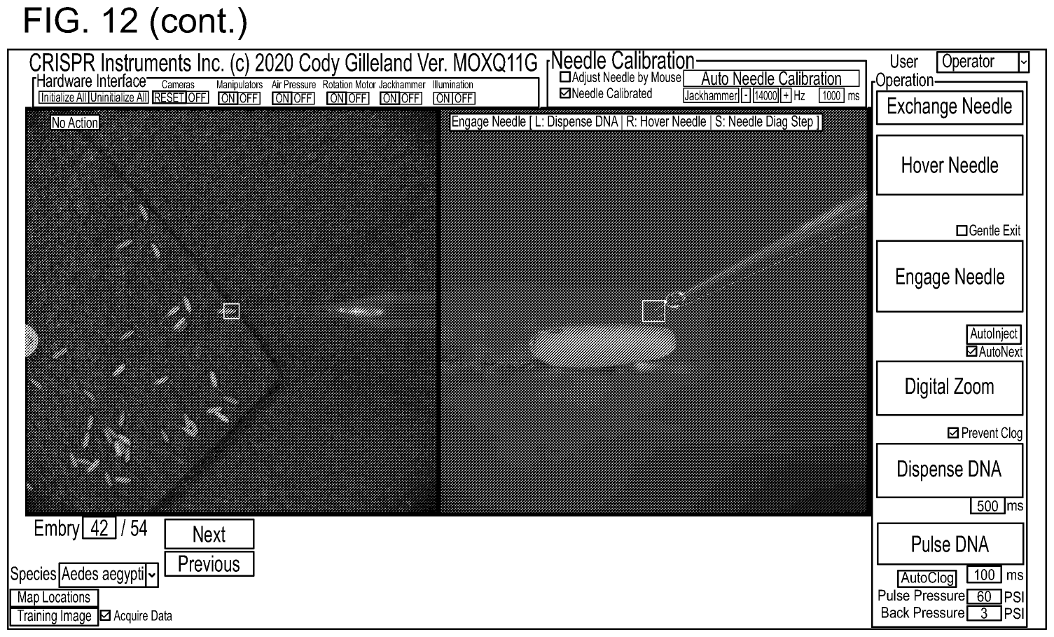

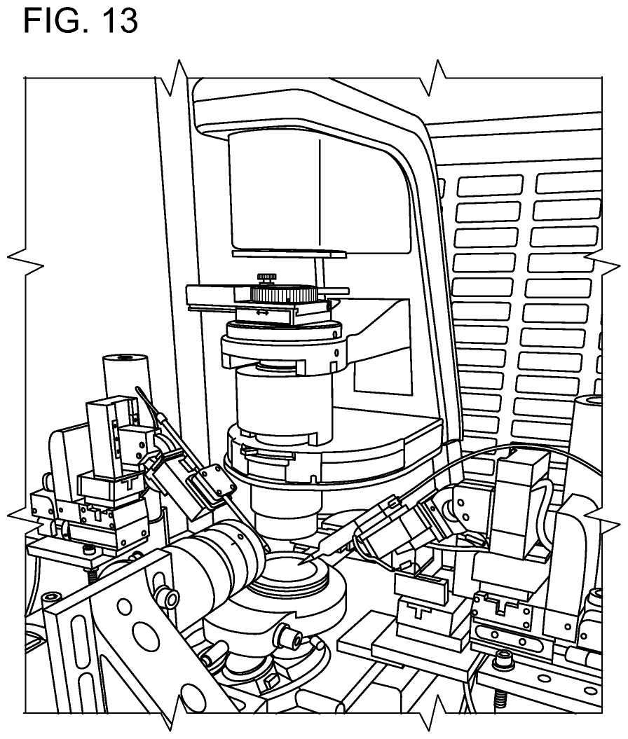

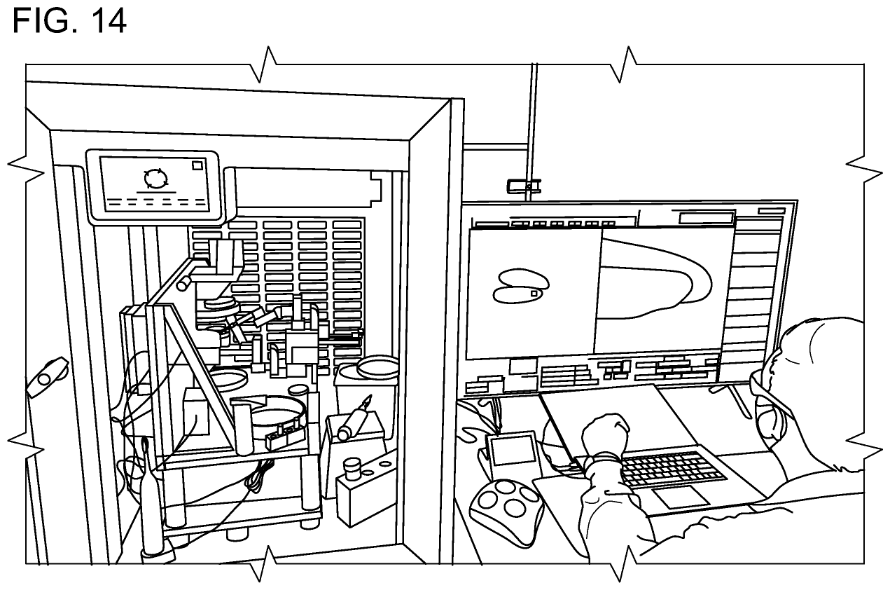

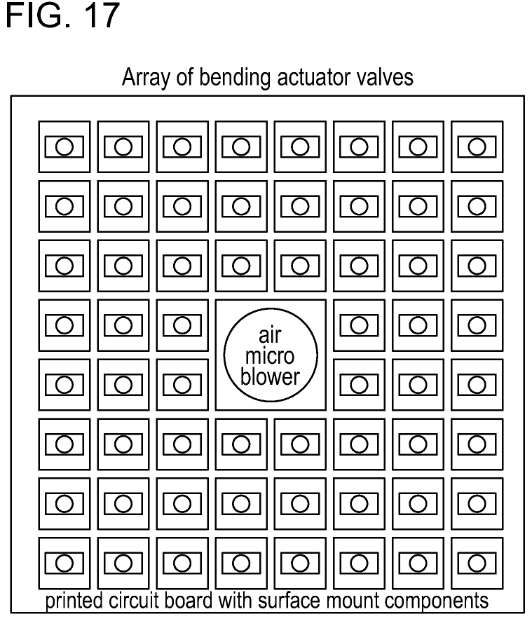

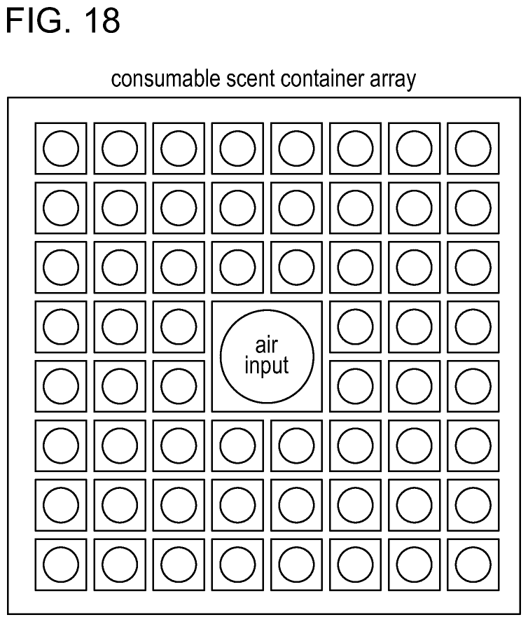

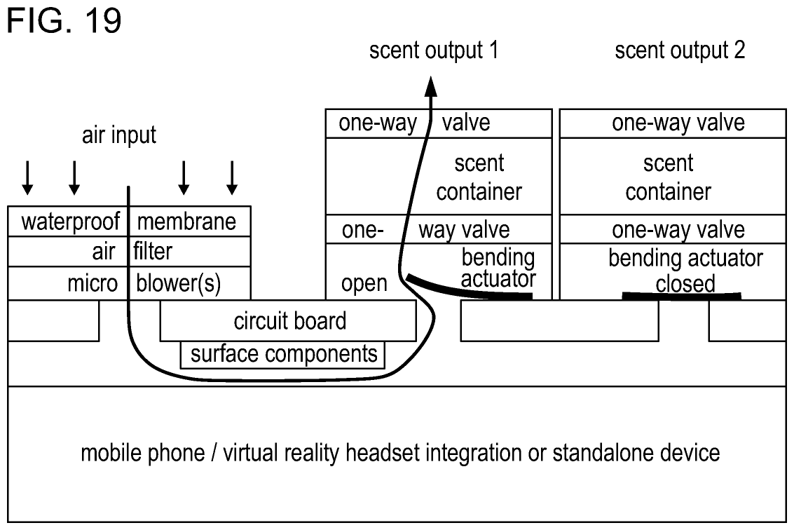

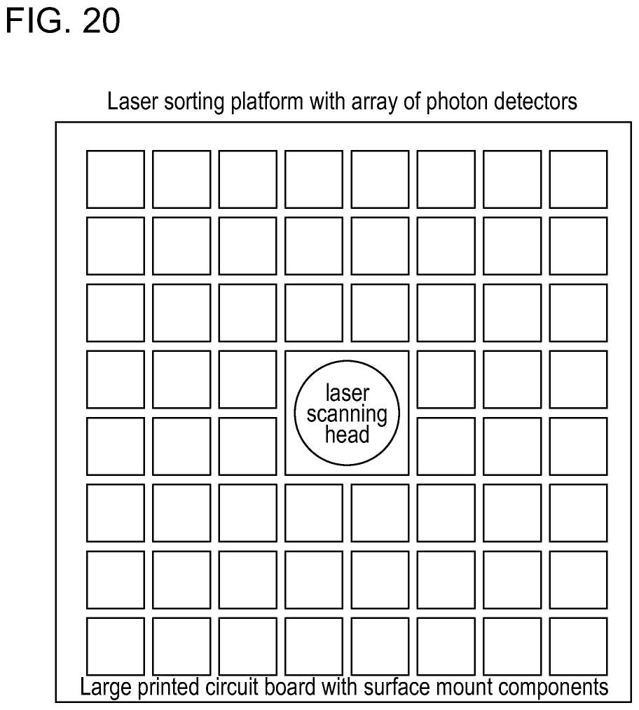

In the present invention the robotic microinjection platform construction is provided in which three cameras enable simultaneous video streaming of high magnification, low magnification camera views with a third horizon view for Z-depth information to enable microneedle alignment. The triple camera method in combination with image processing algorithms enables automated microneedle alignment and organism targeting in low magnification followed by microneedle manipulations of the organism in high magnification. The object detection algorithms are trained using neural networks on large image datasets. The object detection algorithms improve over time as the training dataset grows as data is acquired with more experimentation. The organisms are immobilized in a hydrogel and the sample stage is moved in X, Y, Z, and Theta (angular rotation) directions to position and orient the organism target for microinjection with real-time feedback from high-speed cameras. The lightweight ultrasonic rotation stage enables the sample holder to mount to the XYZ stage without gears or belts to engage and can be dropped in directly from a robotic arm. The stationary multi-view optical design eases manufacturing, enables cost reductions from additional moving parts and enables precision targeting since the object can be located in low magnification then fine-tuned for placement in the high magnification without mechanical drift or delay from changing optical positions. Please see FIG. 1 for a diagram of the electronic control system. Please see FIG. 2 for a diagram of the precision pressure control system. Please see FIG. 3 to describe the arrangement of robotic manipulators for microscope integration. Please see FIG. 4 for the mirror mounting apparatus to enable dual microscope views. The Drawings are FIGS. 1-20 serving purposes as recited in legends, thereto; and incorporated by reference into this specification. The Detailed Description is recited in the following and preceding paragraphs including the legends incorporated by reference.

It is an object of this invention to create a platform that can enable scalable transgenesis without the need for manual dexterity or extensive training while reducing human fatigue through robotic and software algorithm automation under computer control through a software interface. The operation is completely under digital control which allows for remote operation and full automation once adequate training data is collected from user-controlled operations.

The manual organism mounting process is now replaced by the novel process of immobilizing the organisms en masse in a hydrogel to perform precision microinjections of genetic constructs. The hydrogel securely mounts the organism to prevent movement in position as the sample holder is moved in XYZ and Theta directions to autofocus, orient and present the organism target to the microneedle (Z-Autofocus+Theta rotation+XY translation). The organisms can be immobilized for multiple hours without loss of hydration or delays in development. The organism is then recovered by diluting the hydrogel with the organism's preferred media and gently shaking to release the organisms into the media. The mixture of hydrogel and media is then diluted and the organism is transferred to the organism's preferred liquid culture or a plate to continue development.

In the case of organisms that prefer oviposition of embryos in water or mud, such as mosquitoes, the moist hydrogel can be placed directly into a cage of gravid females. We demonstrated that the gravid Anopheles gambiae and Aedes aegypti female mosquitoes oviposit embryos directly into the hydrogel. Gentle robotic vibration causes the eggs to align together and sink more securely into the hydrogel. The Anopheles gambiae eggs are immobilized in the hydrogel and proceed with normal development including melanization and filling of air sacks. After recovery by diluting the hydrogel with deionized water the eggs hatch into the larval stage with full swimming action. Their development does not appear to be hindered in any way. Since all three of the mosquito species including Anopheles gambiae, Aedes aegypti and Culex quinquefasciatus demonstrated survivability after immobilization in the hydrogel we can reasonably expect that this methodology claimed here is extensible to other mosquito species.

The manual embryo alignment process is now replaced by an XYZ & Theta rotation stage with computer vision algorithms to orient and position the hydrogel immobilized organism target within the low magnification field of view camera followed by fine adjustments in the high magnification field of view camera. The rotation stage consists of an ultrasonic ring motor that relies upon friction to engage the drive mechanism, thus the sample holding dish can be placed upon the ultrasonic rotation stage since it does not need to engage gears or belts. Algorithms calculate angular rotation and XY translation distances for visual servoing under the microscope using robotic movement to present the sample to the microneedle at the desired orientation. The computer program sends an electronic control signal to the microcontrollers of the robotic manipulators to drive the angular and XYZ positioning mechanisms with visual feedback from the low magnification and high magnification camera views.

The manual Z focus adjustment is replaced by our novel autofocus technology that enables the user to automatically bring the organism target into focus within the high magnification microscope lens over large Z distances. This is also used to rapidly select organism targets using trained neural networks on graphical processing units based on large image training datasets. We enable rapid and precise autofocus with single-micron-precision over 20-millimeter range by pushing the hardware limits of precision actuation and detection in combination with software-based curve fitting and smoothing algorithms. The algorithm performs a Z movement sweep up to 20 millimeters while recording nanometer resolution Z-positions, high-speed camera images and nanosecond resolution timestamps from the central processing unit (CPU). An algorithm rapidly processes each image and determines a custom focus score value. The algorithm fits smooth curves to interpolate between data points of Z position and image focus score. These smooth curves of Z-axis and image focus score are plotted in reference to the nanoscale resolution timestamps. The algorithm selects the peak focus score (or detected target by image processing and neural network) and correlates this peak value on the smooth curve with the timestamp of the image when the sample was in the optimal focus position. This timestamp is then used to select the Z-position where the organism was in the optimal focus. The robotic nanomanipulator then rapidly moves to the selected Z-position with optimal focus in the high magnification field of view camera.

In contrast to the current state of the art where the flexure guide is located external to the piezoelectric actuator which requires glues and external tubing connected to the actuating end of the microneedle (causing off-axis movement). In the presented invention the metal flexure guide is located internal to the piezo enabling physical support and complete sealing of high-pressure airflow without wear or outgassing of chemical glues. The metal flexure provides a rigid guide along the diagonal axis of microneedle penetration with minimal parasitic off-axis movement. This metal and ceramic-only design enables this piezoelectric penetration unit to be heat sterilized without physical deformation drift or degrading the integrity of the high-pressure seal. The parts are metal 3D printed in stainless steel to enhance the design for manufacturability and ease of assembly. The screw threads are then tapped to ensure tight seals between each part. The solid ceramic is designed with a bore in the middle to allow the metal flexure to be inserted in the middle of the ceramic to provide the on-axis high frequency microscale movements for jackhammer penetration. The electrical connection wires are embedded into the rear metal mounting part to prevent off-axis movement. This unit is then securely mounted to the diagonal axis of the microinjection manipulator for operation. This piezo jackhammer is then controlled using a high frequency signal generator to actuate using the piezoelectric effect. The voltage and drive frequency can be adjusted to accommodate different sized piezoelectric units and resonant frequencies of the load of the microneedle mount on the actuating end of the piezoelectric element. Feedback sensors on the piezo in combination with high-speed camera detection with image processing under the high magnification objective are able to detect the optimal resonant frequency for maximum on-axis penetration and minimal off-axis drift during actuation. The frequencies and voltages are each swept to find the optimal parameters for actuation.

In contrast to solenoid valves, our piezoelectric pneumatic valves enable dynamic and precise pressure level regulation under strict temporal control from a microcontroller analog signal with rapid real-time video feedback from image processing algorithms using high-speed cameras and graphical processing units (GPU). This enables a programmable pressure wavefront to gently fill the organism without the damaging water hammer effect. The combination of microcontroller and piezoelectric pneumatic controller enable the creation of any arbitrary shaped pressure pulse that can be customized to fit the target organism. A programmable smooth Gaussian pressure wavefront delivers adequate amounts of fluid while minimizing the internal damage to the organism. If the algorithm detects a clogged or reduction in fluid flow then the system shall automatically perform high-pressure pulses to remove the clog or alert the operator to exchange the needle if the clog cannot be removed. The algorithm automatically adjusts the pressure levels in response to small changes in the needle opening as large-scale microinjections are performed.

In contrast to manually manipulating the organisms by hand, a robotic arm moves the sample dish holder under a cooled hydrogel dispenser then places a dish with hydrogel into a cage with gravid females. The gravid females oviposit eggs directly into the moist hydrogel. After oviposition, computer controlled positive and negative air flow removes adult organisms that are adhered to the hydrogel surface. The robotic stage moves the sample dish to a suction tip targeting by camera image any remaining adult females adhered to the hydrogel surface.

The grating allows the eggs to pass through while gently lifting out any adult mosquitoes that may become adhered to the hydrogel surface. The hydrogel surface is chilled from below to remain in liquid state. Once the adult mosquitoes are removed using the grating the hydrogel dish is vibrated to allow eggs to settle into the gel. Once the eggs are settled the hydrogel is warmed from below to transition into a more hardened gel state to immobilize the eggs for microinjection.

The ultrasonic ring motor rotation stage engages by friction and does not need belts, gears or lubrication allowing the robotic arm to drop the sample holder dish directly into the microscope XYZ rotation stage. The robotic arm also lifts the sample dish holder after microinjections are completed and transfers to the recovery where media is automatically dispensed to dilute the hydrogel and release the organisms. This allows multiple transfers of freshly oviposited organisms to be rapidly microinjected at early developmental stages to reduce mosaicism. This automated organism loading and immobilization process enables the rearing cages and microinjection platform to be in an enclosed environment without human interaction for safety and security from escaping organisms. A carousel of rearing cages can be rotated to introduce fresh gravid females for access by the robotic arm. A computer-controlled air blade prevents organisms from escaping as the electronically controlled cage door is opened.

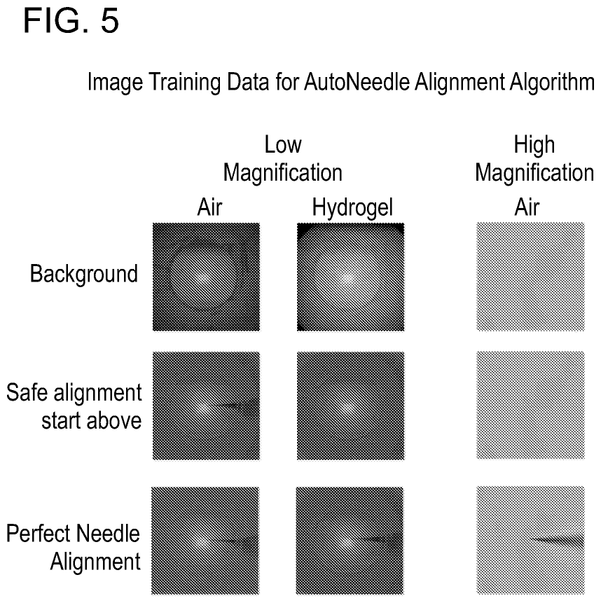

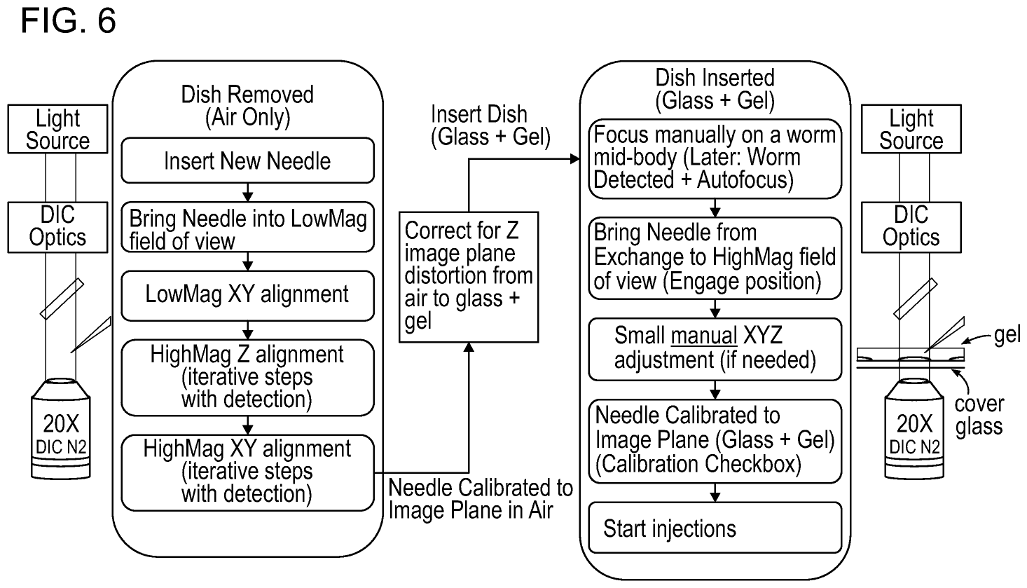

The software interface enables simultaneous low magnification (view organism locations), horizon mid-range magnification (needle alignment in Z, protects breakage and overshooting), and high magnification camera lens views (precision targeting where needle is aligned to image plane) at high frame rates. The operator does not need to mechanically change an objective to view a different magnification. This enables rapid targeting and operation without objective position calibration shifts associated with changing objectives. The software platform can be operated remotely over the internet to enable both training, operation and support. A single button initializes all hardware and software. A single button automatically performs microneedle alignment to the high magnification camera lens as in FIGS. 5 and 6. The piezoelectric penetration element is automatically activated at the resonant frequency as the microneedle approaches the organism target. The piezoelectric actuator operates at high frequencies to delicately penetrate through tissues and outer casings of organisms. To assist with penetration and targeting with computer vision-based feedback from high-speed cameras the needle position may be adjusted by computer algorithm by moving on-axis in 10 nanometer increments to reach the desired location. The user may also incrementally control the needle position by scrolling the mouse wheel to penetrate through flexible tissues or outer casings. A Gaussian pressure wave is initiated with a real-time high frame rate feedback to control the fluid flow. Once a threshold based upon changes in camera frames during the fluid flow is reached the pressure is gently ramped downward to prevent damage from the water hammer effect from rapid pressure changes. Pressure pulses are controlled by a software and firmware-based microcontroller electronic circuit output to a piezoelectric pressure regulator with fast response times on the order of 10 milliseconds. After delivering fluid pulses the nanomanipulator is then retracted slowly on-axis and then the speed increases once the microneedle has exited the organism. A microcontroller signal controls the nanomanipulator to slowly and gently remove the needle after microinjection to prevent the organism's internal contents from spilling out to enhance organism survival rates. A single button on the interface moves the nanomanipulator with microneedle to the home position where the needle can be exchanged and a new sample can be loaded.

Recent advances in spatiotemporal control of gene editing technologies using wavelengths of light to activate or inactivate desired components. The system implements a light that presents spatiotemporal control light-activated or light-inactivated wavelengths. The light can activate that may be lethal at an earlier stage.

Integrated laser with illumination path via 50/50 mirror to kill unwanted mosquito embryos to prevent excess screening using exposure to high intensity laser energy. The intensity can be reduced to uncage reporters or activate/inactivate gene editing technologies.

The delivery of gene editing constructs into fish embryos at the single cell stage proves difficult as the embryo floats freely inside the perivitelline space making microinjection into the single cell nucleus very difficult. The described invention adds a second nanomanipulator to (1) delicately penetrate the chorion using a piezoelectric jackhammer, (2) orient using fluid flow and (3) then stabilize the embryos for (4) microinjection into the single cell nucleus. This process must be completed quickly within approximately 30 minutes before cellular divisions can increase the rate of mosaicism.

The multi-barrel quartz pipette is connected to piezoelectric flow generators with ˜10 millisecond pulse resolution enabling precise rotation of embryos to enable positioning for optimal injection angle of desired target location. The hold position is implemented when all four of the barrels pull a vacuum to immobilize the embryo for micromanipulation. A microcontroller sends signals for small iterations in movement with feedback from a microscope camera with control feedback algorithm for rapid, delicate and precise organism orientation. (Right) The algorithm enables a direction of rotation by pushing a positive pressure while pulling a negative pressure to create a gentle flow of fluid to rotate the embryo. Between each ˜10 millisecond rotation step signal input the embryo is held in place by pulling a vacuum from all four of the multibarrel openings. This enables approximately 1 degree of embryo rotation per step event.

The anti-vibration hardware enables the microneedle to perform microinjection motion of 1 mm travel to the image plane with no detectable vibration or overshoot in the high magnification camera with sub-micron resolution. The X-axis overshoot and Y-axis orthogonal vibration is less than 1 μm in each axis. The metal mounting components are designed in 3D modeling software, machined from aluminum and black anodized to coat the part for electrical isolation. The injection needle holder rod is held between two identical mounting pieces. The piece is held in place by 4 screws with an air gap that can be compressed to hold the rod and minimize vibration. The ultrasonic ring rotary motor holder is attached to the manipulator stage with screws that extend into a ring to support the rotation motor. A 1 mm layer of foam tape is used to allow the high frequency vibration to successfully operate to produce the rotation waves to move the custom rotary stage using constructive microscale friction forces at resonant frequencies.

The dynamic air pressure control employs a high-speed microcontroller over with custom firmware to enable fast air pressure switching capable of performing microinjections. Electromagnetic solenoid valves can fail over time due to degradation from high electrical currents and resulting high temperatures that wear on the metal coil. Most solenoid valves can only provide an ON or OFF position which leads to rapid changes in pressure similar to a square wave inside the organism during pressure pulses producing strong cavitation waves inside delicate internal tissues. During improvements we move from an electromagnetic solenoid valve to piezoelectric pneumatic actuation for extreme reliability and precision. This allows the pressure value to be controlled dynamically within 10 milliseconds and shaped pulses can be produced by controlling the analog input signal designed in firmware. This system can also be used for rodent tail vein injection that synchronizes with rodent heartbeat to dispense reagents during the diastolic phase of the heartbeat. The air pressure source for lower pressure systems could also use a piezoelectric microblower paired with a check valve (silicon valve) that allows one-way flow with low activation pressure and strong reverse flow resistance. The dynamic air pressure control process in the complete system enables unclogging and delicate pressure delivery in the form of a smooth Gaussian wave for gentle fluid delivery.

The software interface has been integrated with firmware to control the microinjection platform using ultrasonic manipulator, air pressure controller, and cameras. In the supplementary slides, a video demonstrates the robotic platform performing a microinjection under software control using C. elegans. The user clicks on the software interface to move the worm in XY and focus in Z by scroll wheel. The user then clicks to engage the needle to penetrate the C. elegans cuticle and gonad sheath. The user pulses the plasmid pressure to deliver the fluid into the gonad region. The user retracts the needle and moves to the next worm in low magnification followed by fine positioning in high magnification. All operations are performed using the software interface and do not require the user to lean into a microscope significantly limiting user fatigue.

The system operates within a covered enclosure for operator safety that helps to maintain the temperature at an optimum level for hydrogel stability in the event that lab temperature and humidity fluctuates. It also includes the following features: (1) HEPA filter to prevent dust particles from clogging needles during plasmid loading/needle installation, (2) Transparent ¼ inch safety polycarbonate protects user in case of needle breakage and projectile at 100 PSI, (3) Access point at back to allow access to electrical control lines and air pressure line, (4) Metal doors open to allow needle loading with plasmid and installation to manipulator mount, (5) The desktop computer and power supplies are on a cart below to isolate fan vibration and heat, (6) Adjustable height standing desk enables user comfort for extended operations, (7) Closed-loop temperature controller regulates air temperature and flow, (8) Humidity control with relative humidity sensor feedback loop. Piezoelectric water vapor generation is directed at the hydrogel surface and driven by ultrasonic frequencies that are inaudible to human ears.

Safety switches to quickly turn off air pressure and robotic manipulator control are built into the software. Software buttons are clearly labeled on the interface and easily accessible. The air pressure is controlled by a 24V power supply and 0-10V analog input signal generator of a Controllino based on the arduino platform with extended ports for analog, high voltage relays and digital outputs. The “Air Pressure OFF” button signal is pressed by the user and the serial communication port is closed to prevent accidental high-pressure pulsing (up to 100 PSI). Prior to closing the serial communication port, the microcontroller firmware provides analog signal to leave the pressure on as the constant gentle backpressure (˜3 PSI) to prevent tip clogging from biological debris or hydrogel drying to the tip. While the communication port is closed the pressure controller is completely disabled from operation for user safety. As the user selects the function to exchange the needle for a new one (“Exchange Needle” button) the pressure is automatically disabled for safety allowing the user to load a new needle into the injection manipulator. After the automated needle alignment algorithm, the user must click the “Air Pressure ON” button to enable the high-pressure pulse feature during operation.

The ease of use of the software interface is paramount to scalability by technician operation. This platform has been successfully and independently operated by three different technicians. The technology platform has operated for 30,000 microinjections without interruption for 25 months resulting in the generation of hundreds or transgenic nematode lines. Rapid and precise microinjection with high experimental certainty and clear evaluation of results enable rapid design-build-test cycles. This is enabled by remote support capability where the software interface can be live streamed to phone or laptop for live support. Technicians are trained remotely on the software interface during normal production experiments and recorded training videos before arriving at the laboratory to accelerate the training cycle.

This additional hardware, circuitry and firmware enables delicate penetration of nematode cuticle, insect chorion, mammalian zona pellucida and rodent tail vein. It also works synchronously with micromanipulator movement to activate during needle penetration but turns off after penetration to prevent overheating of electronic driver circuit hardware and unnecessary heating due to high frequency friction (+/−1 μm@8 KHz) inside the organism gonad region. The frequency is tuned for resonance to prevent off-axis vibration. This can be done using camera feedback or a resonance-locking circuit.

The challenges of loading a new needle limit scale since broken or clogged needles need rapid replacement and alignment to the high magnification objective with small viewing window and short focal depth. Manual alignment is slow with a high failure rate and can result in damaged optics as the needle can be crashed into the objective. The platform implements a dual or triple camera view to simultaneously view the needle in low magnification, high magnification and side view of the horizon to get Z depth without changing objectives. This simultaneous high and low magnification view enables precision needle alignment and sample targeting without moving objectives that can lead to small shifts in alignment, simplifies manufacturing and enhances system reliability with no moving optical parts. Algorithms then detect the needle and move it in XY and Z to bring it into the field of view of the high magnification objective for calibration. The needle is calibrated in air (no glass dish+gel) and then automatically calibrates to be directly in focus when the dish+gel is then loaded with the nematodes. Please see FIGS. 5 and 6 for detailed descriptions of the automated microneedle alignment algorithm workflow and implementation.

The quartz microneedles have been optimized for the smallest, sharpest tip to minimize damage but are still able to flow fluid into the organisms. Every needle is pulled with a perfectly sharp and open tip. It does not require the user to break open the tip as with other needle designs which can result in large tips that can cause excessive damage. The tip opening is ˜500 nm and is used at 60 to 100 PSI to delicately fill the organisms with the desired fluid mixture.

A high-resolution, low-magnification camera is used to enhance the automatic needle alignment algorithm to 100% accuracy for dozens of trials. This 12.4-megapixel sensor achieves 12-bit resolution for significant gray levels to detect the microneedle tip from the background. Mapping locations of organisms in low magnification is important for locating the targets for microinjection in high magnification. This camera is based on a silicon scientific CMOS sensor with high dynamic range enabling the detection of the needle tip in lighting conditions meant for the DIC microscope in high magnification. Please see slides to describe the algorithm in detail with experimental images. In summary, the image of the organism is selected from a larger area image of multiple organisms by thresholding and sorting large articles within the area size constraints relating to adult nematodes and embryos of insects and fish. For nematodes, once the single organism is selected a spline is drawn down the “spine” of the organism using a traveling branch algorithm while shorter branches are iteratively pruned in favor of one single long branch down the center as the spline. Distances are measured at 25% of the total length from each end to indicate the gonad region target area for further inspection in the high magnification camera. Once each gonad location is mapped the waypoints are created and the XY stage moves in a nearest-neighbor-approximation algorithm to visit all targets until the desired injections are completed. For mosquito embryos, the orientation is detected using image processing by analyzing the shape of the egg and determining the sharpest curvature to determine the posterior end of the embryo. The desired approach angles and XY locations are determined algorithmically to present the posterior end of the embryo for microinjection. The algorithm has the ability to prioritize embryos based upon the stage of melanization by detecting the level of total grayscale in the embryo outline in the low magnification camera view.

We have developed the fastest and most accurate image-based autofocus technology that enables acquisition of a long distance 200 μm Z-stack with results down to a single micron of precision. The process takes ˜1 second and travels longer Z distances while using only the camera and XYZ stage manipulator. There are no additions to hardware that can be expensive and add complexity. This has been released to production mode and successfully completed at least 30,000 microinjections with no failures. High-speed cameras work synchronously with precision robotics and powerful GPU processing to accurately sweep a 200-micron Z-stack and return to the Z position of highest focus score within a single micron of accuracy in ˜1 second. The timestamps for each image and robotic Z position are read directly from the computer processing unit (CPU) with nanosecond resolution. The data is then processed by filtering, interrogating and smoothing to find the most accurate focus position. Please see FIG. 6 for description of the Autofocus algorithm.

The hardware to dynamically and safely adjust the needle pressure to a maximum pressure of 100 PSI has been implemented to enable 30,000 microinjections. A software-controlled, robust piezoelectric valve dynamically regulates the pressure from 0 to 100 PSI with +/−0.5 PSI accuracy and 10 millisecond response time. The needle holder and piezo jackhammer are designed to withstand 100 PSI while only flexing +/−1 microns in XYZ dimensions allowing the needle to remain in place during high-pressure pulses over long duration to clear a clogged needle. This allows the use of a piezoelectric jackhammer penetration component to gently enter the organism followed by high-pressure pulses without needle position distortion while inside the delicate organism. This allows the use of small needles on the order of 500 nm for microinjections. The smaller needles flowing high-pressure pulses in combination with constant backpressure (˜3 PSI) prevent needle clogging from biological debris. This algorithm is necessary for mosquito microinjection since clogged needles can interrupt the ˜45 min window where the embryos must be injected before they melanize and undergo cell division that could enhance undesired mosaicism. The Controllino uses custom arduino-based firmware to send the analog input control signals to the piezo-pneumatic regulator to produce the high-pressure pulse. The user presses the “AutoUnclog” button on the software interface and the pressure automatically increases from ˜3 PSI backpressure to maximum of 100 PSI for 1 seconds to clear the clog. The software algorithm uses the camera to detect the fluid flow from the needle and dynamically adjusts the pressure level for the next injections. If there is no fluid flow then the high-pressure pulse is repeated two additional times before notifying the user to change out the clogged needle then use the AutoNeedle Alignment algorithm. This process enables the user to return to production microinjections within 1 minute in total and significantly reduces the need for needle exchanges by unclogging needles with extended high-pressure pulses.

The gonad XYZ target is selected in high magnification and the algorithm engages the needle to penetrate the worm cuticle with the piezo jackhammer. The algorithm detects the initial image, delivers a pressure pulse and then takes a second image. The images are then subtracted to find the difference between them. This process is repeated for up to 4 times as the needle retracts along the diagonal axis of injection ˜3 microns each iteration. Once a minimum fill threshold is met the algorithm automatically stops and gently retracts the needle. If a pulse does not produce any detectable fluid delivery, then the pressure is automatically increased by 1.2×, 1.4×, 1.8×, 2× for each of the 4 iterations with a maximum of 100 PSI. If the injection filling exceeds the max threshold, then the next injection starts at 50% of the previously selected pressure in the next organism injection to prevent overfilling and damaging the organism. Images of the algorithms of the processing the gonad filling images are included in the supplemental slides.

The ultrasonic rotation stage enables the sample to be oriented for microinjection in a tightly constrained area while providing illumination from the top for DIC imaging with imaging from below. There are no moving parts, gears to engage, or lubrication that may impact sterility or lead to contamination. Ultrasonic nanoscale vibrations work together to rotate a custom machined dish holder. The ultrasonic motor is silent since it operates at high frequencies above the audible range for humans. Complexity is shifted to robust electronic circuitry and firmware. This algorithm is necessary for mosquito microinjection since the eggs must be oriented for microinjection into the tail end. In traditional manual microinjection methods, one person is manually orienting the mosquito embryos with a paintbrush or tweezers while the other injects. The ability to find the desired XYZ locations while determining the angle of orientation using computer vision followed by robotic XYZ Rotary motion. This methodology allows one person to operate the microinjection platform and enhances the survival rate since the eggs are not handled and the microinjection should be less damaging. Since the user does not have to wait for mosquito eggs to melanize for manual alignment the embryos can be injected earlier in the developmental cycle while the chorion is more elastic with fewer nuclei to edit for large CRISPR knock-in insertions and knockouts.

Remote support and operation via live streaming of the software interface to a PC or smartphone. The entire operation has been digitized and under software control enables a remote operator to control the platform for training, support, and production scale operation by remote employees. This enables the operator to be in a different location and future employees can be trained and brought up to speed prior to platform hardware arrival to the new location. Streaming the software interface to a smart phone enables immediate support to assist with live troubleshooting while an employee is away from a computer. The streaming platform is enabled by wifi internet or high-speed mobile data streaming. The platform shall make use of the most recent technology available for high-speed data including 4G, 5G and future data streaming protocols.

The delicate recovery of organisms after microinjection in combination with the correct temperature to enable nuclease cutting and DNA repair for CRISPR knock-in and knockout activity. For nematodes, the platform demonstrates a new rapid plate shaker can agitate the hydrogel at room temperature to release the nematodes without chilling the gel to release them from the hydrogel. The computer-controlled shaker is tuned to operate at 750 RPM with 1 mm radius of rotation. We demonstrate that allowing the Cas9 protein and RNA complex to remain at higher temperatures enhanced the Cas9 activity. In this case the CRISPR insertion was an mTurquoise marker that can be screened in a fluorescence microscope. We performed 90 microinjections in 22 minutes and then recovered using the new rapid computer-controlled shaker at room temperature. This resulted in 13 large sequence confirmed CRISPR insertions that were 2.4 kb in size. The system consistently produces about 900 F1 transgenics per set of ˜50 injections across multiple co-injection markers.

The software interface has been optimized for successful use at scale by 3 different technicians. Feedback from users include requests for larger buttons with space in between to prevent inaccurate clicking during rapid operation. The operating system has been deployed as an executable file to maximize use of CPU and GPU resources. We integrated a camera with high dynamic range to enhance automated microneedle alignment and enhanced the software algorithm to enable hundreds of successful operations without error.

For mosquito transgenesis, current manual methods require the technician to wait for the chorion to melanize and become hardened before they can be manually manipulated to align them for microinjection. They must also be dehydrated to allow more fluid to enter since the chorion is hardened. We demonstrate the use of a moist, temperature-sensitive hydrogel to immobilize embryos to remove the step of manual handling which often results in damaged embryos. We demonstrated that Aedes aegypti mosquito can oviposit eggs directly into our hydrogel that is placed into a cage of gravid females. This enables us to gently immobilize the embryos without human handling for the first time. Since we are able to immediately immobilize and place the dish into the transgenesis platform we are able to begin microinjections before they have melanized. In current manual methods the chorion is hardened and does not allow large volumes of fluid to enter the embryo. In the case of pre-melanized microinjections the chorion expands significantly to accept large volumes of fluid to allow more Cas9 protein and guide RNA complex material to enter the embryo at an earlier developmental stage.

Transparent microneedle tips can be difficult to visualize and locate against a dark or opaque background or during the penetration step of microinjection. Increasing the camera exposure, dynamic range, gain and image contrast to visualize the microneedle tip can result in excessive bleaching of the target organism of interest. The microneedle shall be illuminated with a laser line sheet to enable simultaneous imaging of the target organism and the microneedle tip. The present design includes a laser line sheet at an angle of incidence to the capillary shaft to enable laser beam propagation to the needle tip. This causes the tip to emit laser light as a glowing beacon for out-of-plane detection where short depth-of-focus objectives can detect needle locations even when the placement is out of the limited focal depth. A laser emits a shaped line in parallel to the imaging plane to ensure that the capillary shaft is illuminated in any location of the imaging plane. The continuous wave laser can also be pulsed at predetermined frequencies to distinguish laser tip illumination from background noise. For reliability, stability, low heat dissipation and user eye safety from low wattage (Class II, less than 1 mWatt, ˜520 nm wavelength) operation the design can include a direct-diode semiconductor laser that does not require a crystal that can cause heat dissipation, energy inefficiency and part complexity. The electronic driver is connected to the arduino based microcontroller firmware with electrical signal relay to control the blinking laser pulses through the software interface. The laser line sheet emission angle is limited to the robotic travel area of the needle to prevent unwanted laser scattering and is directed at absorptive material for safety.

The laser pulse illumination also enables the user and image processing algorithms to detect if the needle tip has successfully punctured through the outer casing of the organism. In this case we consider an organism outer casing to be nematode cuticle, insect chorion, mammalian/fish zona pellucida. As the needle penetrates through the opaque outer casing. This increase in body illumination just after penetration also enables the detection of fluid pulse size for video feedback into the real-time piezo-pneumatic controlled fluid delivery algorithm. While some laser light can emit through the chorion before penetration significantly more light fills the internal body cavity after penetration that can be easily detected by image processing and comparing the differences between the current and previous images in the video stream. This algorithm operates iteratively by pulsing the piezoelectric jackhammer and moving the robotic manipulator forward along the diagonal needle penetration axis. The algorithm then compares the image differences in the pulsing illumination to detect penetration by body illumination. The algorithm has real-time feedback where the step cycle repeats iteratively until penetration is detected with an imposed cycle limit to prevent needle breakage from over-traveling or excessive organism damage. The microscope illumination can be reduced or turned off and the camera exposure and gain increased to detect the full body laser illumination to detect microneedle penetration. Low power low heat laser is necessary to prevent damage to the organism and the injection mix. A gentle back pressure prevents needle clogging from biological debris while the gentle, smooth Gaussian pulse flow enables gentle fluid flow into the organism. The high-speed cameras take averages of multiple images to reduce image noise from water microdroplets being dispensed onto the sample from an ultrasonic mist generator to maintain sample moisture.

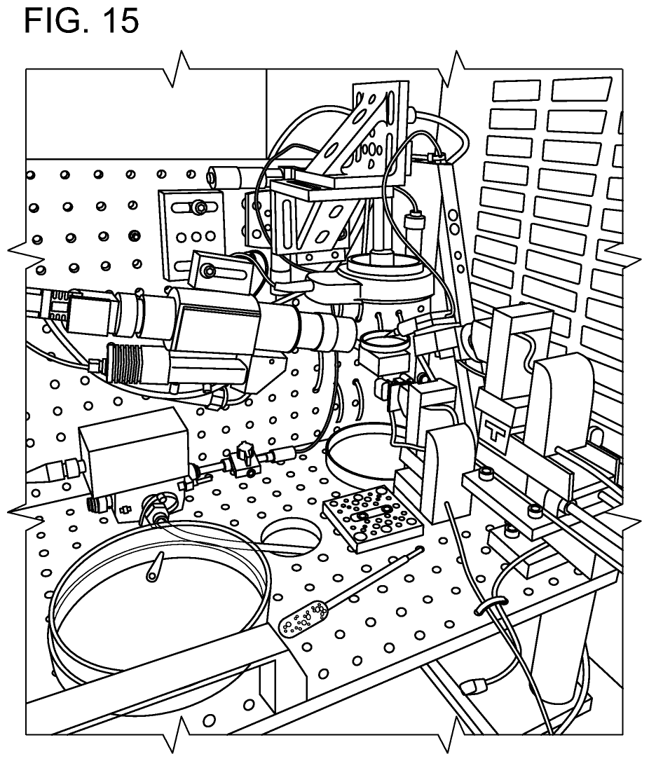

The upright microscope arrangement (FIG. 15) primarily targeting insect embryos includes 3 different cameras and lenses for simultaneous imaging and algorithmic processing in real-time. The long-distance microscope lens is oriented at the horizon to view at about 10 degrees from the horizon to see the side view of the sample during micromanipulations, microinjections and microneedle alignment. The low magnification views the sample area to determine the XY locations and desired rotary position of the target samples to orient them to the microneedle. The upright high magnification lens possesses a long working distance for micromanipulation operations and a short focal depth for precision targeting. The upright and horizon lens arrangements can also include in-line illumination. For reliability there are no moving parts in the microscope and the organism sample is positioned for imaging and targeting with assistance from computer vision and machine learning algorithms trained using image-based datasets. Each of the high magnification, low magnification and horizon camera views are streamed simultaneously at high-speed and fed into algorithms for image processing. For example, the low magnification view determines the XY position and the horizon view in combination with a rapid autofocus algorithm determine the correct Z target location to select the injection target. The piezoelectric rotation motor orients the sample with computer vision feedback. The lenses can be outfitted with liquid lenses for rapid autofocus applications and remote optical alignment after shipment for installation.

The inverted microscope arrangement primarily for organisms that require differential interference contrast (DIC) imaging to visualize sub-organism targets. This includes an inverted DIC microscope with illumination condenser, polarizers and analyzers with short working distance high magnification objectives. An in-line 50% transmission, 50% reflection mirror (50/50 mirror) is mounted just below the condenser in a custom-machined mount to enable a low magnification view with telecentric lens. This optical configuration enables minimal disruption to the linear polarization of the DIC optics while enabling accurate telecentric XY positions information to be acquired in low magnification. For sensitive imaging applications the mirror coating percentage can be reduced significantly to prevent image distortion or angled adjacent to prevent interrupting the illumination source from the condenser. A macro lens or long-distance microscope lens is directed at the 50/50 mirror to view the sample area from the top in direct alignment with the high magnification objective to determine sample XY target locations and XY needle location to enable microneedle alignment algorithms. The laser line illumination is useful to visualize the glowing microneedle tip as the short focal depth and short working distance make the alignment process susceptible to microneedle breakage against expensive high magnification optical lenses. In this arrangement the piezo rotary motor has a low profile with wide internal bore to allow DIC optics to pass through completely.

The mammalian embryo culture dish is a mobile element that continuously stays with the specific embryos and is tracked with blockchain, radio frequency identification and consists of sensors to measure temperature, humidity, oxygen, carbon dioxide with real-time series data and tolerance algorithms for sending alerts. These sensors could also be implemented on a single chip or circuit board for miniaturization and low power consumption with integrated batteries for reliability. Culture media is circulated and refreshed using extremely low power piezo microblowers or pumps for automated embryo culture. Small enclosed units enable conservation of environmental gases and precision control of each microculture system.

Production Scale Transgenesis: These systems have been successfully reduced to practice and used in production scale to inject 90,000 nematodes (C. elegans, C. brenneri, C. briggsae) and 60,000 mosquito embryos (Aedes aegypti, Aedes albopictus, Culex quinquefasciatus, Anopheles gambiae). In addition, we demonstrate microinjection of housefly (Musca Domestica) and Asian citrus psyllid (Diaphorina citri) which are notoriously difficult to microinject. We also produced transgenic lines of the traditional fruit fly model organism, Drosophila melanogaster, and the agricultural pest Drosophila melanogaster. The flexibility of our software interface enables a single mode option to be changed to adapt each setting to be changed between organisms to adapt to level of camera exposure, digital zoom, microinjection depth and penetration procedures, pressure wave profiles, and organism detection algorithms. This organism selection mode option can be changed automatically by system as it detects the organism using image-based algorithms.

Nematode Efficiency Data: Each set of C. elegans injections consistently produces about 600 F1 transgenics from 40 microinjections using an mScarlet pan-neuronal co-injection marker. The platform generates CRISPR insertions in C. elegans with high efficiency where 90 microinjections in 22 minutes of system operation demonstrates 14 successful large CRISPR insertions (2.4 kb mVenus fluorescence marker). We show a more than 30× improvement in transformation efficiency over manual method with C. briggsae (nematode related to C. elegans). This organism is much more difficult to microinject manually due to their delicate nature. The trained technicians are able to operate the platform to produce 300 F1 transgenics with 40 injections in comparison to 10 or less F1 from manual injections. C. briggsae survive at higher temperatures (29° C.) which enables the study of gene editing nucleases that are more active at higher temperatures. Our ability to bring a 30× efficiency boost in C. briggsae shows promise for enhancing transgenic and gene editing efficiency of other delicate organisms where manual microinjection may be rate-limiting or intractable as in crop pest nematodes that infect roots of soybean plants, corn and other staple crops.

Mosquito Efficiency Data: We demonstrate microinjection into Aedes aegypti and Culex quinquefasciatus in the pre-blastoderm state while the chorion is clear and most flexible to enable greater filling of RNA+Cas9 complexes. Efficiency data in Aedes aegypti show 51% survival and 58% knockout rate. The results show 58 adult survivors out of 113 embryo injections produced 24 surviving females with 14 showing doublesex knockout phenotype giving a 58% knockout rate. We also demonstrate knock-in CRISPR insertion capability in Aedes aegypti by producing a red body phenotype in just 22 microinjections, a near 10-100× improvement over manual methods due to extreme robotic delicacy and removing the need to manually handle them for orientation. This enables earlier delicate injections before cell division occurs giving better access to germline cells for editing.

The novel optical design enables the user to simultaneously visualize embryos and needle interface in high magnification as they are clustered together. The simultaneous high magnification and low magnification views enable rapid microneedle alignment algorithms and ability to locate targets without changing optical lens. A telecentric macro lens or long-distance microscope lens with short focal depth is directed just above the horizon to view the sample area from the side about 100 degrees from the high magnification objective to determine the needle Z height and Y location to enable microneedle alignment algorithms. The upright telecentric lens enables visualization of the entire field of view to detect embryo/organism XY locations. Computer vision and machine learning algorithms detect the embryo/organism positions and calculate the optimal rotary angle of injection and XYZ location. For example, in Aedes aegypti the algorithm detects the anterior/posterior ends of the embryos and stores the XY locations optimal approach angles in relation to the stage encoder positions. Rotary and XY movements position embryos for the microinjections then fine corrections and completed with digital zoom turned on to precisely position the embryos with computer vision algorithms. The embryo is first positioned in XY and optimal approach angle with the upright low magnification view followed by XZ position correction in high magnification from the horizon view.

Mammalian embryo cloning and gene editing can make use of new capabilities to generate transgenic lines using methods such as base editing, prime editing that can be introduce by pronuclear injection while monitoring the filling of the nuclear envelope. The piezoelectric vibration unit enables penetration of the zona pellucida and nuclear envelope with minimal invagination and damage to embryo structure. The piezoelectric vibration and diagonal on-axis microneedle movement is done in small incremental steps (˜1 micron distance, 50 millisecond delay) to prevent heating from mechanical vibration and enable compute time for real-time computer vision algorithms to assess the current position and target location. Each incremental step requires approval from the algorithm to proceed, thus enabling precision targeting and preventing damage to the embryo. The Gaussian pressure wave delivery system is trained to deliver a gentle pulse of fluid containing genetic material into the nuclear envelope to measure a ˜10% increase in volume using computer vision algorithms. Cloned into a cell line, deep sequence confirmation with whole genome coverage with long reads for deletions.

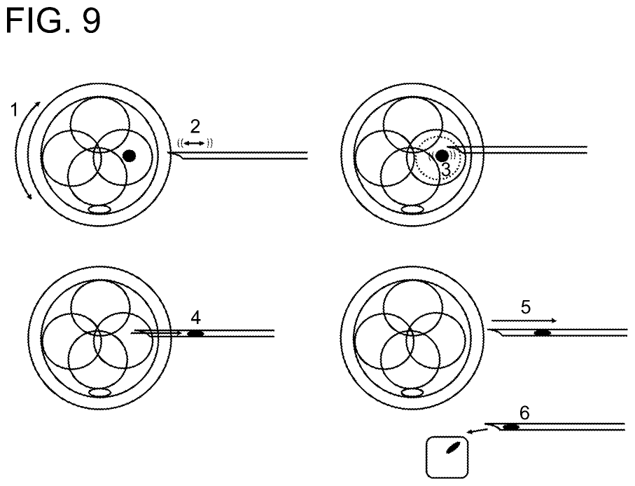

Mammalian embryo biopsy and enucleation requires the use of manual methods that remain rate-limiting despite decades of improvements in genetic technologies. The user can select the embryo orientation and the desired biopsy location by clicking on the software interface. The biopsy needle can then extract penetrate using piezoelectric vibration and delicately reach the desired location of extraction of nuclear DNA, mitochondrial DNA, and related cellular contents (FIG. 9). The user can scroll backward on the mouse scroll wheel to aspirate the cellular materials by triggering a vacuum in pulsed steps. The computer vision algorithm or user controls the interface by point-and-click to locate and approach the embryo target in low magnification to bring the target within the field of view in high-magnification. The algorithm or user the orients the embryo for access the desired point or needle insertion for insertion of sperm (ICSI), single cell biopsy, and multi-cell biopsy. The algorithm the pulses the piezoelectric vibration unit for on-axis penetration through the zona pellucida and into the inner cell contents. The algorithm or user then elects to perform the desired operation for dispensing sperm, disrupting the internal cell contents followed by extraction for analysis. Depending on the mode the user shall be restricted to prevent dispensing of a biopsy before it is in the analysis container location.

An algorithm for collection and deposition of cellular material for analysis is enabled by a rotating ring of collection tubes that bring the collection device into the travel range of the precision robotic manipulator system for each sample to the collected from the microneedle by pressurizing the inside of the needle. The internal cell contents are then deposited and monitored to ensure the contents are properly collected and then sent for analysis.

For future pandemic preparedness the ability to rapidly generate and scale clonal populations of gene edited mice with human targets of interest could accelerate the vaccine and antibody response. For example, the human ACE-2 receptor cloned from a cell line into a transgenic mouse for rapid trials at scale. The goal is to prevent the delay from scaling up a mouse colony and the genetic from breeding. Eggs and sperm could be frozen and a wild-type mouse colony could be maintained as preparedness requirement.

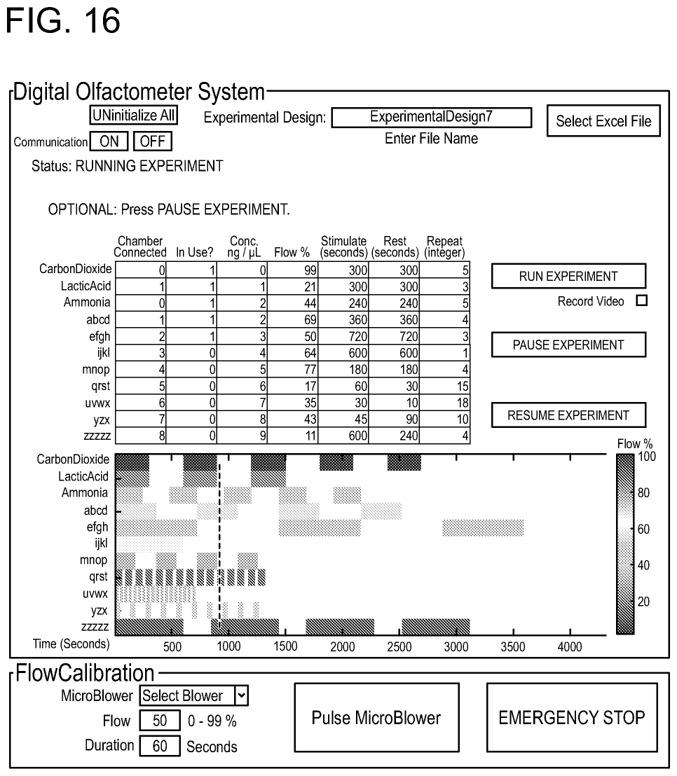

Precision control of fluids and air flow can enable digital olfactometry for sensing and behavior response to stimuli. Our system enables the user to create a test pattern in an excel file and upload it into the software interface. This interface can also be used for rodent tail vein injections to delicately dispense fluids in response to the low-pressure diastolic sequence during rodent heartbeat. This heartrate stimulus can be detected by electrical signals from the mouse heart or visually from a fast camera with motion amplification algorithms. A microcontroller then sends the drive signal to the pressure source with microsecond resolutions. This could be done with a piezo-pneumatic regulating drive blower to enable precision dosing. Please see FIG. 16 for the software-controlled user interface of the microdosing platform.

The present device relates to the fields of olfactory stimulation and precision drug delivery. Technologies for digital sound using electronic speakers and visual stimulation using screen pixels convey audio and visual natural landscapes, movie scenes and music; however, the olfactory system is untouched from a digital standpoint. Current scent distribution technologies have leakage or do not enable fast time resolution of scent signals. The goal of the present invention is to bring digital, fast spatio-temporal control of multiplexed scent array without leakage in addition to temperature and humidity control. The design of the system lends itself to miniaturization, multiplexing, and IP65-rated waterproofing with ultra-low power requirements enabling portability with battery and integration with existing mobile devices including smart phones.

The large area printed circuit board design for culturing synthetic biology product ferments and harvests in small continuous batches to optimize for microdosing packaging. The aim of optimization targets enhancing input to product yield by using small batches made continuously and in a massively parallel operation. Analogous to how a compute server farm operates through parallelization and redundancy this style of operation is the strategy to prevent having to optimize for large tanks that may decrease yield and compromise product quality. This ensures a high-quality product can be made without risk of loss and remove human intervention that may introduce contamination and reduce the use of contamination sanitization chemicals. The printed circuit board employs surface mounted electronics for detection of culture conditions (pH, temperature, oxygen, optical cell density turbidostat) and microdosing of reagents for even distribution and temperature control. This design prevents the need for large steel bioreactors, manual labor for installation, optimization for large scale cultures and reduce foam from dead microbes that reduced yield because conditions were not homogenous throughout the large bioreactor. This design enables full robotic control and harvesting and local production close to the consumer.

Manufacturing and Assembly Innovation: The key to scalable and economical commercial use is the simplified design for manufacturing process and innovative array design with surface mounted electronics with reliable solid state piezoelectric vibration for both atomization of the desired scent or chemical and the piezoelectric microblower to move the air to user's nose or lungs in a precision microdosing capacity for digital aerosolized drug delivery in a multiplexed format. This strategy also leads to greater power efficiency and robustness of the device to mechanical stresses during consumer use. By using surface mounted electronics onto a printed circuit board coated in resin this enables the process of integrating the wireless controllers, electronic signal generators and signal conditioning components (voltage boost, amplifiers, clocks, precision timers, frequency controllers, sensors). The design and manufacturing of the printed circuit board with surface mounted electronics enables full robotic automation of the mounting process through pick and place handlers and solder reflow lending itself to the economies of scale enabled by. The piezoelectric atomizer units (piezo sheet and micro-holes sheet) are manufactured in an array and mounted directly to the printed circuit board for control signal input. The low power consumption of piezoelectric components enables further miniaturization. The piezoelectric atomization is manufactured from a sheet of ceramic material with electrically conductive film on both sides. A laser scanning instrument is used to cut the desired shape of the piezoelectric array of concentric circles but leaving enough material to provide mechanical stability. The laser scanning instrument can then be used to create micro-drilled holes in the metal substrate to provide holes for atomization of liquids based upon desired hole size and density to produce the desired output. The laser-drilled micro-holes can be further optimized for the volatility, viscosity and other fluid dynamic parameters of the specific atomized product. A program is created and the target sheet is recognized with computer vision for targeting, mapping and alignment of the desired drilling pattern. The contactless nature further reduces costs of equipment maintenance and variability of tooling instruments as they lose sharpness over extended use. One side of the conductive piezoelectric sheets shall be used as common electrical ground for each unit of the array. The other drive signal side shall have the conductive electrical film laser-drilled to produce an electrical lead pattern to interface each array unit to the corresponding mapped drive signal input mount that leads into the interface to the printed circuit board. The solder provides both electrical and mechanical stability from the drive piezoelectric and the. Using a single sheet piezoelectric device and metal sheet for micro-drilled holes provides uniform planarity for interfacing the consumable swappable unit with the surface mounted electronics on the printed circuit board surface. The ground side of the piezoelectric sheet securely mounted with magnetic material. The magnetic material sheet then mounts to the individual array units to provide the mechanical coupling drive connection to the atomizing metal sheet with laser micro-drilled holes. This magnetic mounting strategy enables rapid swappable changes without contamination of leakage of the chemical contents. The piezoelectric unit shall remain stationary to the printed circuit board and the laser drilled micro-hole array sheet with desired chemicals shall be the swappable unit to refresh or change out the consumable inputs. The board mounted side provides electrical conductivity to the printed circuit board to provide the input drive signal and can be securely mounted using a solder bump array. The solder bump array also enables scalable testing by probe card design using pogo pins to test the electronic circuit board and surface mount electronics before the piezoelectric mechanical vibration drive array is attached. This assembly step is enabled by surface mount pick and place automation of the entire array sheet at once and solder paste reflow to connect each piezoelectric array drive component to the array mapped with the. There is an electric insulating adhesive layer between the ground side of the piezoelectric drive element and the magnetic sheet to protect the metal sheet from electrical noise and current leakage. The affordable nature of the swappable consumable is enabled by simple and low-cost manufacturing of the metal sheet with laser micro-drilled holes and a well array to contain the consumable chemicals. Further the consumable chemicals can be using high throughput liquid handling arrays. Piezoelectric liquid handling can enable rapid filling of the well array with precision volumes. A peelable film is used to protect the surface of the atomizing head of the consumable and is peeled directly before installation.

In sum, a method is provided for delicately introducing genetic material into organisms by immobilizing, orienting and positioning organisms for precision microinjection and intracellular component extraction through a microneedle capillary with computer algorithm feedback from simultaneous low magnification and high magnification microscope with high-speed cameras.

As a further aspect of the invention, a method is provided comprising the simultaneous low magnification and high magnification microscope by placing a beam sampling mirror in the optical path that allow a portion of the light to pass through to the high magnification objective while reflecting a portion of the optical path to allow a simultaneous low magnification and high magnification view for organism and sample targeting and microneedle alignment using image processing and human-computer interface input.

As a further aspect of the invention, the upright microscope arrangement has the mirror in the path between the condenser light source and the sample with enough clearance for the micromanipulators to access the sample in focus of the high magnification objective.

As a further aspect of the invention, the sample is a nematode, fish, insect, plant, mammalian embryo or tissue. Where the sample is a single cell to be extracted or injected.

As a further aspect of the invention, the robotic XYZD+Rotary manipulators use ultrasonic motors that lock in place for stability when not in motion to maintain precision and prevent movement jitter as with electromagnetic AC motors.

As a further aspect of the invention, the organism is embedded in a temperature sensitive pluronic hydrogel inside a glass holding dish to reversibly and securely immobilize the organism while providing hydration, temperature and humidity control.

As a further aspect of the invention, where a custom grating restricts the XY locations where insect embryos can be oviposited and provides a filter for allowing the embryos to remain in the hydrogel while removing adults that may adhere to the hydrogel surface.

As a further aspect of the invention, the software imposes a safety distance limit on the number of iterative diagonal microneedle movements toward the glass surface to prevent needle breakage by the user.

As a further aspect of the invention, the piezo components are assembled without glue and provide mechanical load compression on the piezo unit for stable high frequency operation and 3D printed metal or machined internal flexure guide for on-axis motion and high-pressure capability.

As a further aspect of the invention, the sterilization is monitored by an infrared thermal camera and analyzed by computer vision and machine learning classifier to determine adequate sterilization by image, temperature distribution, pressure and time series of each data point.

As a further aspect of the invention, the apparatus and method have a second nanomanipulator is provided with a plurality of micropipettes to penetrate the chorion using piezo-jackhammer vibration, orient the embryo with fluid flow to allow targeting of the single-cell nucleus then hold the embryo in place with gentle suction for microinjection with first nanomanipulator.

As a further aspect of the invention, the apparatus consists of a pre-fabricated frame from a single piece of metal using 3D-printing or machining to properly align all optical and robotic components during initial assembly to enhance manufacturability.

As a further aspect of the invention, the apparatus is designed to enable remote operation to enable support, training and uses a hardware key for security on both networked computers.

As a further aspect of the invention, the apparatus and method is motion controlled by small increments in piezoelectric steps is monitored by high-speed cameras with machine learning algorithms that approve each motion step to enhance safety and error reduction.

As a further aspect of the invention, the apparatus and method enable automated microinjection penetration by actuating the piezoelectric jackhammer unit under software-controlled signal generator just before coming into contact with the organism and stopping once the microneedle has successfully penetrated determined by image processing.

As a further aspect of the invention, the apparatus is incubated at the optimal physiological condition for each specific organism regarding temperature, relative humidity and oxygen, carbon dioxide and nitrogen gas levels.

As a further aspect of the invention, a laser line sheet is positioned at incident angle to the capillary shaft to propagate to the tip providing illumination to the needle tip for ease of visualization due to contrast enhancement enabling robust image processing of tip location and penetration into organism as the organism body illuminates.

As a further aspect of the invention, a camera and object detection algorithm are used to detect the cellular biopsy contents as they are successfully deposited from the microneedle into the collection receptacle for analysis.

As a further aspect of the invention, the method of disrupting and extracting internal cellular components of a single cell in tissue use directed ultrasonic pressure waves, the method comprising:
a) Immobilizing the tissue in a biocompatible hydrogel at physiological temperature;
b) Selecting a single cell in tissue using computer vision algorithms;
c) Penetrating the cell wall with a microneedle containing a cell culture buffer or incompressible fluid;
d) Disrupting the internal cellular content while preserving its natural state using directed pressure waves from the piezoelectric unit.

As a further aspect of apparatus and method, an ultrasonic transducer is used to produce a pressure wave through a needle tip targeted at a single cell. The pressure waves disrupt the internal cell contents and then to be extracted without clogging the needle by using a hydrophobic coating on the needle to prevent adherence of biological material.
a) Tuning the pressure waves to disrupt the intracellular contents of the cell without disrupting the cell wall;
b) Variable frequencies to avoid standing waves and interference;
c) Extracting the intracellular components that are in a natural form that can be provided.

As a further aspect of the invention, the chamber is sealed to allow the pressure wave to travel through the needle tip, As a further aspect of apparatus and method the system is computer controlled, and, where the specimen is a mouse embryo, fly, zebrafish, nematode, human tissue, human tissue organoid, human embryo, the ultrasonic pressure wave of liquid is used to puncture the mammalian zona pellucida, where the system has integrated vision feedback for targeting, extraction, microinjection, where the system is dynamically controlled with pressure inputs, where the sample is extracted and deposited into a well, and the sample is processed using PCR, where the sample may be RNA-seq, the sample may be genome sequenced, where the sample may be exome sequenced.

As a further aspect of the invention, the apparatus and method of the needle is cleaned using a heat source, the heat source is a laser, a ceramic infrared heater, is a flame, is acoustic, is a heated air or thermal camera detects heating profile to confirm sterilization.

As a further aspect of apparatus and method a training algorithm processes thermal images to confirm sterilization.

As a further aspect of apparatus and method the optical system has an upright telecentric low magnification lens with a horizontal lens elevated above the floor plane with long working distance and shallow focal depth for visualizing needle interactions.

As a further aspect of apparatus and method the automated workflow consists of sample targeting using machine learning and computer vision trained with image datasets.

As a further aspect of apparatus and method the auto-inject microinjection workflow consists of sample targeting, high-pressure pulse clearing of microneedle, manipulator on-axis needle movement, piezoelectric vibration for penetration entry, gentle piezo-pneumatic pressure wave of injection mix, on-axis needle retraction, high-pressure pulse clearing of microinjection needle.

As a further aspect of apparatus and method the mammalian cloning workflow consists of robotic movements and image detection of a cell, microneedle piezo-vibration penetration of cell to access the nucleus, aspiration of the nuclear material, orientation of the enucleated embryo, penetration of the zona pellucida and cell wall, of delivery of foreign nuclear material.

As a further aspect of apparatus and method the optical system consists of a simultaneous dual view high magnification objective lens with long working distance and shallow focal depth and low magnification objective lens with no moving parts and digital zoom for image enlargement.

As a further aspect of apparatus and method the software interface enables complete remote operation to align microneedles, position/orient the target samples and perform micromanipulations/microinjections by clicking on software interface and with computer vision and machine learning algorithms.

As a further aspect of apparatus and method the software interface has a simultaneous low magnification and high magnification views.

As a further aspect of apparatus and method the optical and robotic components quickly and economically assemble through magnetic mounts and are aligned using algorithms with integrated electrical connections to the mounting hardware.

As a further aspect of apparatus and method the mounting hardware is 3D printed and assembled with magnetic mounts, integrated high frequency electronics on custom printed circuit board and anti-vibration stabilization with active electronic control and passive damping.

As a further aspect of apparatus and method needle mounting is clamped by piezo ring facing inward perpendicular to the needle that is loosened by applying voltage and pushed out by high pressure pulse and remains securely tightened at rest)

As a further aspect of apparatus and method the pressure and extraction pulses are ultrasonic for only 10 ms at a time to prevent heating of the cell. High intensity and low duty cycle (like femtosecond laser surgery).

As a further aspect of apparatus and method the platform is connected to the cloud for algorithms and data analysis.

As a further aspect of apparatus and method the platform can detect and extract cancer stem cells to discover new genomic targets.

As a further aspect of apparatus and method the platform can detect and extract immune cells targeting cancer stem cells (metastasizing cells, immune cells).

As a further aspect of apparatus and method the clog is broken up and pushed out using ultrasonic pressure pulses.

As a further aspect of apparatus and method the clog is burned using intense laser heat and ultrasonic pressure waves.

As a further aspect of apparatus and method the reagent is loaded into the needle by a microfluidic filter device or acoustic droplet.

As a further aspect of apparatus and method the needle is cleaned (by laser heat and ultrasonic waves) and patch/clamp pipette can be reused for scalable experiments.

As a further aspect of apparatus and method the electrical properties of the needle can detect a glass surface coated with ITO to detect height and prevent crashes into the glass surface.

As a further aspect of apparatus and method the embryo, biopsy sample, and data including genomic, temperature, images are location tracked with radio frequency identification chip, physical barcode and blockchain in real-time workflow reports to prevent error of misplacing or erroneously swapping embryos and eliminate failure of culture conditions.

As a further aspect of apparatus and method insect larvae, pupa and adults can be sorted by fluorescence marker or morphology using computer vision and machine learning training datasets.

The organisms have distinct size differentiation the male and female can be bulk sorted by wind resistance by dropping organism into an air current and sorting by larger cross-sectional area using distance traveled in the presence of a uniform air current. A conveyor belt with scooping treads lifts organisms from liquid culture, distributes by vibration and continuously transports them through a wide area multicamera, multi-lens fluorescence imaging system and targets unwanted organisms with a scanning laser that rapidly kills unwanted organisms for applications of screening for transgenics post-injection and scaling sex sorting for absolute fidelity cage trials and field deployment. A rapid scanning ultraviolet laser at low intensity can be used to excite green fluorescent proteins over a large area approximately 1 meter square to raster an image using silicon photomultiplier devices (SiPM) in an array on a custom printed circuit board to detect ultra-low levels of fluorescence. The XY laser scan location in real-time that requires no delay in processing because a custom photodiode circuit can be triggered to increase the intensity of the laser to kill when a fluorescence signal is detected with nanosecond and micrometer resolution. The rapid XY scanning is done at such a fast rate and low intensity that the organism is not damaged until the fluorescence signal threshold is detected and the kill signal is activated by high-speed closed-loop circuitry.

The accelerometer determine motion and modulates the scent output signal to adjust for walking versus stationary.

The algorithm can adjust the PWM signal, voltage and duty cycle.

The printed circuit board can be provided with through holes enable the dispensing devices to mount the electrical drive connections directly to the piezoelectric elements. This enables an array of swappable scent units making the system modular, extensible and customizable to the application.

The unit device can be comprised of a combination of piezoelectric substance atomizer, piezoelectric microblower fan to distribute and a series of one-way valves to restrict leakage unless actuated by the drive signal.

The system of above is in a multiplexed plurality of units to create customized environmental scents and scents that are unique to the specific application of interest.

Other objects, features, variant embodiments can be made within the scope of the above described method and apparatus limited only by the scope of the claims recited hereinafter or as per claims and amendment forms, if any.

1. A method for delicately introducing genetic material into organisms by immobilizing, orienting and positioning organisms for precision microinjection and intracellular component extraction through a microneedle capillary with computer algorithm feedback from simultaneous low magnification and high magnification microscope with high-speed cameras.

2. A method according to claim 1, further comprising the simultaneous low magnification and high magnification microscope by placing a beam sampling mirror in the optical path that allow a portion of the light to pass through to the high magnification objective while reflecting a portion of the optical path to allow a simultaneous low magnification and high magnification view for organism and sample targeting and microneedle alignment using image processing and human-computer interface input.

3. A method for miniaturization and manufacturing of a digital microdosing platform employing surface mount electronics on a printed circuit board to enable multiplexed digital microdosing of scents and reagent chemicals using piezoelectric microblowers to produce a flow of air through a water and particle filter that is then routed by an array of piezoelectric bending actuators that actuate to lift and direct air flow through a swappable consumable array containing the individual scents between two one-way valves that prevent leakage while enabling scent production with spatiotemporal control enabled by cryptocurrency microtransactions via scent marketplace enabling real-time entertainment, gaming, advertisements, drug delivery, learning and memory enhancement and olfactory diagnostics for early viral testing, olfactory rehabilitation, neurodegeneration screening and prevention by stimulating olfactory system to enhance neural connections.

4. A method for rapid sex sorting of organisms by fluorescence detection using laser scanning at low intensity and then detecting fluorescence photon signal using a highly sensitive silicon photomultiplier device array coupled with the laser intensity control by amplifying transistor circuit to increase laser intensity to kill with nanosecond time resolution. The system can be set to select for fluorescent or non-fluorescent organisms and scan as a mesh conveyer belt moves organisms.

5. The method of claim 1 wherein the upright microscope arrangement has the mirror in the path between the condenser light source and the sample with enough clearance for the micromanipulators to access the sample in focus of the high magnification objective.

6. (canceled)

7. The method of claim 1 wherein the robotic XYZ manipulators use ultrasonic motors that lock in place for stability when not in motion to maintain precision and prevent movement jitter as with electromagnetic AC motors.

8. The method of claim 1 wherein the organism is embedded in a temperature sensitive pluronic hydrogel inside a glass holding dish to reversibly and securely immobilize the organism while providing hydration, temperature and humidity control.

9. The method of claim 1 wherein a custom grating restricts the XY locations where insect embryos can be oviposited and provides a filter for allowing the embryos to remain in the hydrogel while removing adults that may adhere to the hydrogel surface.

10. The method of claim 1 wherein the software imposes a safety distance limit on the number of iterative diagonal microneedle movements toward the glass surface and disables certain interface buttons during robotic movements to prevent needle breakage by the user.

11. The method of claim 1 wherein the piezo components are assembled without glue and provide mechanical load compression on the piezo unit for stable high frequency operation and 3D printed metal or machined internal flexure guide for on-axis motion and high-pressure capability.

12. The method of claim 1 wherein the sterilization is monitored by an infrared thermal camera and analyzed by computer vision and machine learning classifier to determine adequate sterilization by image, temperature distribution, pressure and time series of each data point.

13. The method of claim 1 wherein the system of claim #1 has a second nanomanipulator with a plurality of micropipettes to penetrate the chorion using piezo-jackhammer vibration, orient the embryo with fluid flow to allow targeting of the single-cell nucleus then hold the embryo in place with gentle suction for microinjection with first nanomanipulator.

14. The method of claim 1 wherein the apparatus used for its implementation comprised of a pre-fabricated frame from a single piece of metal using 3D-printing or machining to properly align all optical and robotic components during initial assembly to enhance manufacturability.

15. (canceled)

16. The Method of claim 1 wherein the apparatus used for its implementation comprised is motion controlled by small increments in piezoelectric steps is monitored by high-speed cameras with machine learning algorithms that approve each motion step to enhance safety and error reduction.

17. The method of claim 1 wherein the apparatus used for its implementation comprised of enables automated microinjection penetration by actuating the piezoelectric jackhammer unit under software-controlled signal generator just before coming into contact with the organism and stopping once the microneedle has successfully penetrated determined by image processing.

18. (canceled)

19. The method of claim 1 wherein a laser line sheet is positioned at incident angle to the capillary shaft to propagate to the tip providing illumination to the needle tip for ease of visualization due to contrast enhancement enabling robust image processing of tip location and penetration into organism as the organism body illuminates.

20. (canceled)

21. A method of disrupting and extracting internal cellular components of a single cell in tissue using directed ultrasonic pressure waves, the method comprising:
Immobilizing the tissue in a biocompatible hydrogel at physiological temperature;
Selecting a single cell in tissue using computer vision algorithms;
Penetrating the cell wall with a microneedle containing a cell culture buffer or incompressible fluid;
Disrupting the internal cellular content while preserving its natural state using directed pressure waves from the piezoelectric unit.

22. (canceled)

23. (canceled)

24. (canceled)

25. The method of using in the implementation of the method of claim 1, wherein the automated workflow consists of sample targeting using machine learning and computer vision trained with image datasets that are acquired as the initial user operates the plat.

26. The method of using in the implementation of the method of claim 1, wherein the auto-inject microinjection workflow consists of sample targeting, high-pressure pulse clearing of microneedle, manipulator on-axis needle movement, piezoelectric vibration for penetration entry, gentle piezo-pneumatic pressure wave of injection mix, on-axis needle retraction, high-pressure pulse clearing of microinjection needle.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. The method of claim 1, wherein the embryo, biopsy sample, and data including genomic, temperature, images are location tracked with radio frequency identification chip, physical barcode and blockchain in real-time workflow reports.

44. The method of claim 1, wherein insect larvae, pupa and adults can be sorted by fluorescence marker or morphology using computer vision and machine learning training datasets. A conveyor belt with scooping treads lifts organisms from liquid culture and continuously transports them through a wide area multicamera, multi-lens fluorescence imaging system and targets unwanted organisms with a scanning laser that rapidly kills unwanted organisms for applications of screening for transgenics post-injection and scaling sex sorting for high fidelity cage trials and field deployment.

45. The method of claim 1, wherein a scent can be paid for or earned using microtransactions of cryptocurrency for payment in a scent marketplace and advertisement by product placement.

46. The method of claim 1, wherein an array of digitally controlled scents can be used for olfactory rehabilitation after smell loss or prevention of neurodegenerative diseases.
